(19) United States
(12) Patent Application Publication
(10) Pub. No.: US 2012/0016285 A1
(43) Pub. Date: Jan. 19, 2012

(54) CATHETER WITH REMOVABLE EXTENSION MEMBERS

(75) Inventor: Sohail Contractor, Watchung, NJ (US)

(73) Assignee: INTER-NOVATION INC., Watchung, NJ (US)

(21) Appl. No.: 13/179,184

(22) Filed: Jul. 8, 2011

Related U.S. Application Data
(60) Provisional application No. 61/364,651, filed on Jul. 15, 2010.

(51) Int. Cl. A61M 25/16; A61M 1/16

(52) U.S. Cl. 604/6.09; 604/533; 604/6.16; 604/535

(57) ABSTRACT

A catheter for use in accessing the vasculature of a patient and for optionally transferring a volume of blood from a patient to a hemodialysis machine. The catheter includes one or more connectors, which are secured to a transition member of the catheter. The one or more connectors have a threaded securement component for facilitating the securement of a secondary apparatus, such as an extension tube member to the one or more connectors.

### RELATED APPLICATIONS

This application claims priority to U.S. Provisional Application No. 61/364,651, filed Jul. 15, 2010, and entitled “CATHETER WITH REMOVABLE CONNECTORS.”

### TECHNICAL FIELD

The present disclosure is directed to a catheter for use in accessing the vasculature of a patient. In more particular, the present disclosure relates to a dialysis catheter for transferring a volume of blood from a patient to a hemodialysis machine for treatment, the dialysis catheter having one or more connectors for allowing securement to and removal of one or more extension members, which facilitate connection of the dialysis catheter to a hemodialysis machine.

### BRIEF DESCRIPTION OF THE DRAWINGS

FIG. 1 is a perspective view of a dialysis catheter having first and second connectors, according to one embodiment, in which a portion of the dialysis catheter is positioned within a patient.

FIG. 2 is a perspective view of the dialysis catheter of FIG. 1 illustrating removal of first and second extension members utilizing the first and second connectors.

FIG. 3 is a perspective view of the dialysis catheter, according to one embodiment, illustrating a transition member and first and second connectors, which have been covered utilizing a dressing to secure the dialysis catheter relative to the patient.

FIG. 4 is a perspective view of a dialysis catheter, according to one embodiment, being inserted into a large vessel of a patient.

FIG. 5 is a perspective view illustrating a dialysis catheter having first and second connectors and first and second extension members, according to one embodiment.

### DETAILED DESCRIPTION

A variety of dialysis catheters have been developed for use in modern medicine to facilitate dialysis in patients who have experienced diminished or permanently compromised kidney function. Dialysis, also referred to as renal replacement therapy, may be used for sick patients who have suddenly, but temporarily, lost their kidney function. Such temporary loss of kidney function is often referred to as acute renal failure. Dialysis can also be used for more stable patients who have permanently lost their kidney function, which is commonly referred to as chronic kidney disease.

When the patient's kidney function is compromised, the natural removal of minerals and water from the blood is compromised. The kidneys also function as a part of the body's endocrine system producing erythropoietin and calcitriol. Dialysis, or renal replacement therapy, provides an imperfect replacement of kidney function due the fact that dialysis is not designed to replace the endocrine function of the kidneys. Dialysis is adapted to facilitate maintenance of desired composition of a patient's blood, primarily by maintaining a desired equilibrium of water and minerals within the blood. Such minerals can include sodium, potassium, chloride, calcium, phosphorus, magnesium, and sulfate. Dialysis treatments help to maintain desired blood composition through the use of diffusion to provide waste removal from the blood, and through the use of ultra-filtration to facilitate fluid removal from the blood.

In a typical dialysis procedure, a dialysis catheter is introduced into a large vein of a patient. A common site for placement of the distal end of a dialysis catheter is the superior vena cava or the right atrium of the patient. Typically, the superior vena cava placement site is accessed either through a subclavian vein in the patient's chest or an internal jugular vein in the patient's neck region. Oftentimes, either the subclavian or the internal jugular vein is accessed on the patient's right side. Once the tip of the dialysis catheter has been introduced into either the internal jugular vein or the subclavian vein, the catheter is threaded to a desired position within the superior vena cava or other large vessel of the patient. Other sites for placement of the distal end of a catheter include the common femoral vein or external jugular veins.

The dialysis catheter will typically include first and second lumens which facilitate removal of the blood from the patient and then allow return of the filtered blood back to the patient. An arterial lumen (e.g., first lumen) of the dialysis catheter facilitates removal of the blood from the patient while also providing a mechanism for delivering the blood to the hemodialysis machine. A venous lumen (e.g., second lumen) returns the treated blood from the hemodialysis machine back to the patient's vasculature. A variety of types and configurations of catheter tips have been developed for use in connection with typical dialysis catheter apparatus. Such catheter tips provide desired intake through the arterial lumen while allowing advantageous return of blood to the patient through the venous lumen.

Traditional dialysis catheters also include a proximal hub which provides a mechanism to separate the arterial lumen from the venous lumen into separate arms of the device. The separation of the arterial lumen and venous lumen provided by the proximal hub facilitates connection of the dialysis catheter to extension tubing, which is ultimately connected to a hemodialysis machine. The separated arterial lumen and venous lumen are typically labeled in red and blue colors, respectively, so as to clearly illustrate to a practitioner desired connection of the dialysis catheter lumens to the hemodialysis machine.

The configuration of traditional dialysis catheter hubs suffers from a number of deficiencies. First, in the event that the dialysis catheter is to remain affixed to the patient between dialysis procedures, the extension tubing connected to the separated arterial lumen and venous lumens can be somewhat long and cumbersome. As a result, prolonged attachment to the patient's body can be somewhat uncomfortable to the patient. Additionally, in patients who are not fully lucid, such extension tubing may be inadvertently grasped by the patient. As a result, this can increase the potential for damage or injury at the dialysis catheter insertion site or even potentially result in undesired removal of the dialysis catheter from the patient. Long extension tubing can also become contaminated, posing a risk of introducing contamination and/or infection to the patient. Some extension tubing in dialysis catheters further include clamp members and/or luer couplers to connect to the dialysis machine. Such clamp members and luer couplers may become contaminated before, during or at the completion of the dialysis procedure. As a result, such components can also introduce a source of contamination and infection to the patient. After the completion of a procedure, attempts to cover, clean, or otherwise disinfect the extension tubes and the associated components can be difficult and is often not effective.

Patients who have daily or ongoing hemodialysis treatments will often utilize a specialized chronic dialysis catheter which is configured to be left in place between dialysis treatments. A chronic dialysis catheter is typically inserted in a minimally invasive procedure in which a cuff or other component is positioned beneath the patient's skin approximately 3-8 cm. The patient's skin can provide a covering which operates as a barrier to infection. However, such chronic dialysis catheters do not overcome many of the deficiencies of existing dialysis catheter mechanisms and can add substantially to the cost and expense of providing ongoing dialysis due to the surgical intervention required for placement.

The present disclosure is directed to a dialysis catheter for use in accessing the vasculature of a patient and for transferring a volume of blood from a patient to a hemodialysis machine. The hemodialysis machine may be configured to filter the blood and return the filtered blood to the patient through the dialysis catheter. According to one embodiment of the present disclosure, the dialysis catheter includes one or more connectors which are secured to a transition member of the dialysis catheter. The one or more connectors may have a securement component for facilitating securement of a secondary apparatus, such as an extension member, to the one or more connectors.

One embodiment of the present disclosure is directed to a dialysis catheter for use in accessing the vasculature of a patient and for transferring a volume of blood from a patient to a hemodialysis machine. The hemodialysis machine is configured to filter the blood and return the filtered blood to the patient through the dialysis catheter. According to one embodiment of the present disclosure, the dialysis catheter includes one or more connectors which are secured to a transition member of the dialysis catheter. The one or more connectors may have a threaded securement component, such as a luer coupler, for facilitating the securement of a secondary apparatus, such as an extension member, to the one or more connectors.

In other embodiments, the catheter may be a tunneled or non-tunneled infusion catheter. In yet other embodiments, the catheter may be other vascular or non-vascular catheters such as nephrostomy and biliary catheters.

According to one embodiment of the present disclosure, the transition member is secured to an elongate access tube. The elongate access tube can comprise a dialysis catheter tube which is inserted into the superior vena cava or other large vessel of the patient. The elongate access tube can include an intake lumen and a delivery lumen. The intake lumen allows the passage of blood from a distal end of the elongate access tube to a hemodialysis machine. The delivery lumen facilitates the delivery of blood from the hemodialysis machine to the patient's vasculature.

According to one embodiment of the present disclosure, a transition member is secured to a proximal end of the elongate access tube. In one embodiment, the transition member includes a branch component comprising a first arm and a second arm. The first arm is in fluid communication with the intake lumen of the elongate access tube. The second arm of the transition member is in fluid communication with a return lumen of the elongate access tube. In another embodiment, the transition member may be a uniform body without a bifurcated hub. Furthermore, tubing may be connected directly to the transition member without the need for a separate connector.

When the dialysis catheter is disconnected from the extension tubing, a minimal and largely unobtrusive profile relative to the patient is provided in connection with the combination of the transition member and the first and second connectors. In other words, when extension tubing is removed from the first and second connectors, only the transition member and first and second connectors may remain outside the patient. This not only facilitates improved comfort when worn by a patient between dialysis procedures, but also improves health and safety to the patient. For example, by not having elongate tubing extending from the dialysis catheter on the exterior of the patient, the likelihood that a patient will accidentally bump, grasp, or otherwise pull at the portion of the dialysis catheter positioned outside the patient is greatly diminished. In this manner, accidental injury at the hemodialysis site or inadvertent removal of the dialysis catheter from the patient is minimized.

By minimizing or eliminating extension tubing which would otherwise remain connected to the transition member when not in use, any contamination or sources of infection that are associated with the extension tubing, clamp members, or luer couplers secured to the extension tubing can be reduced or eliminated. For example, during a subsequent dialysis procedure, new sterile extension tubing members can be secured to the connectors, reducing the likelihood of the introduction of contaminants or other sources of infection. Additionally, high levels of heparin, which would otherwise remain as residue on/in previously used extension tubing, are not reintroduced back into the patient.

Another advantage provided by the lower profile of the transition member and first and second connectors is that OPSITE™ or another protective covering can easily be placed over dialysis catheter, including the transition member and the first and second connectors. Not only does this protective covering retain the transition member and first and second connectors in a desired placement relative to the patient, but it also helps to maintain a clean and sterile environment between dialysis procedures. The placement of an OPSITE™ covering or other hygienic covering also minimizes the possibility that a less-than-lucid patient might grasp the extension members or another portion of the dialysis catheter, increasing the potential for damage to the catheter access site or result in inadvertent removal of the dialysis catheter from the patient.

According to one embodiment of the present disclosure, first and second extension members are provided in connection with first and second connectors. The first and second extension members may be configured to be threadably coupled or otherwise mechanically coupled to the first and second connectors. The first and second extension members can include extension tubing, clamp members, and/or luer fittings which can be utilized to provide desired operability of the first and second extension members. The luer fittings can facilitate connection of the first and second extension members to, for example, tubing connected to the hemodialysis machine. According to one embodiment of the present disclosure, the first and second extension members may each comprise a threaded collar. The threaded collar can comprise a female threaded component, which is configured to attach to a male threaded component of the first and second connectors. When the first and second extension members are positioned in threaded coupling with the first and second connectors, ongoing flow of blood from the first and second connectors into the first and second extension members is provided. A clamp member associated with the tubing of the first and second extension members can be utilized to control the flow of blood through the first and second extension members until luer fittings on the proximal portion of the first and second extension members are secured to tubing of the hemodialysis machine.

The above-referenced descriptions of the summary of the disclosure are provided for illustrative purposes only. Additional aspects and advantages will be apparent from the following detailed description which proceeds with reference to the accompanying drawings and the appended claims.

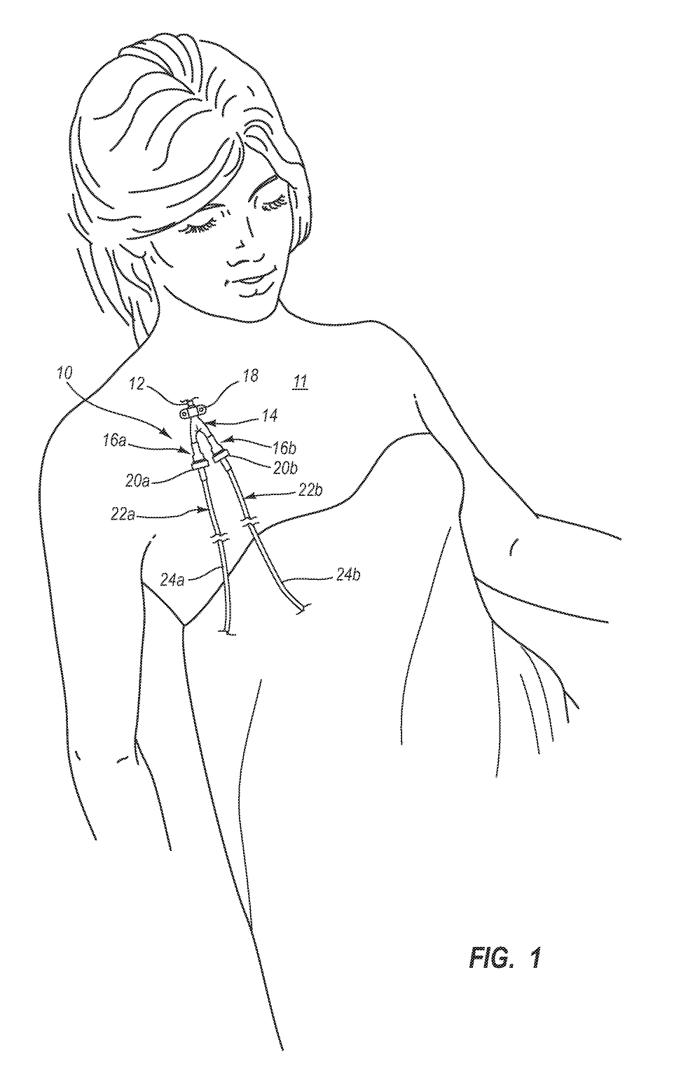

FIG. 1 is a perspective view of a dialysis catheter 10, according to one embodiment of the present disclosure. In the illustrated embodiment, dialysis catheter 10 has been inserted into a chest of a patient 11. Dialysis catheter 10 comprises an access tube 12, a transition member 14, a first arm 16a, a second arm 16b, and a suture bracket 18. Access tube 12 of dialysis catheter 10 is inserted into the chest of patient 11. As previously discussed, access tube 12 provides a mechanism for transferring volumes of blood from a patient to a hemodialysis machine for filtering of the blood. The access tube also facilitates returning of the filtered blood to the patient.

Transition member 14 provides separation between an intake lumen and a delivery lumen of access tube 12. As a result, the single catheter tube provided by access tube 12 is split into first arm 16a and second arm 16b. First arm 16a is in fluid communication with the intake lumen of access tube 12. Second arm 16b is in fluid communication with the delivery lumen of access tube 12. Suture bracket 18 is positioned between access tube 12 and transition member 14. According to an alternative embodiment of the present disclosure, suture bracket 18 is positioned on a distal end of transition member 14. Suture bracket 18 provides a mechanism for securing dialysis catheter 10 to the patient. Suture bracket 18 includes wings positioned on either side of dialysis catheter 10 with an aperture positioned through each wing. In this manner, a practitioner can suture the suture bracket 18 directly to the patient's skin to securely fasten dialysis catheter 10 such that access tube is maintained in a desired position within the patient's vasculature.

In the illustrated embodiment, connectors 20a, b are provided at the proximal portion of first arm 16a and second arm 16b. Connectors 20a, b allow connection of extension members 22a, b to dialysis catheter 10. Extension members 22a, b can include or be coupled to extension tubing 24a, b which are connected to a hemodialysis machine. By providing connectors 20a, b in connection with a dialysis catheter, extension members 22a, b can be secured to components of dialysis catheter 10. For example, extension member 22a and extension tubing 24a can be secured in fluid communication with first arm 16a to facilitate delivery of blood from an intake or arterial lumen of access tube 12 to a hemodialysis machine. The hemodialysis machine can then be utilized to filter and otherwise treat the blood. Once the blood has been treated by the hemodialysis machine, it can be returned to the patient through extension tubing 24b, extension member 22b, second arm 16b, and then from the delivery lumen or venous lumen of access tube 12 to the patient.

As will be appreciated by those skilled in the art, a variety of types and configurations of dialysis catheters can be utilized without departing from the scope and spirit of the present invention, as claimed. Various catheter types and tip designs may be used including split tip or step tip designs, infusion catheters, access catheters, non-tunneled catheters, and the like. According to one embodiment of the present disclosure, the dialysis catheter can be secured to the patient utilizing a mechanism other than a suture bracket. According to another embodiment of the present disclosure, the connectors can be secured to the transition member utilizing tubing rather than being secured directly to the transition member. According to yet another embodiment of the present disclosure, additional features can be provided in connection with the extension members. For example, according to one embodiment of the present disclosure, luer fittings can be positioned at the proximal portion of the extension members to permit securement and removal of extension tubing to the extension members. According to another embodiment of the present disclosure, clamps can be provided on the tubing of the extension members. According to yet another embodiment of the present disclosure, the extension members do not comprise part of the dialysis catheter but instead can be provided separately as disposable additions to the dialysis catheter.

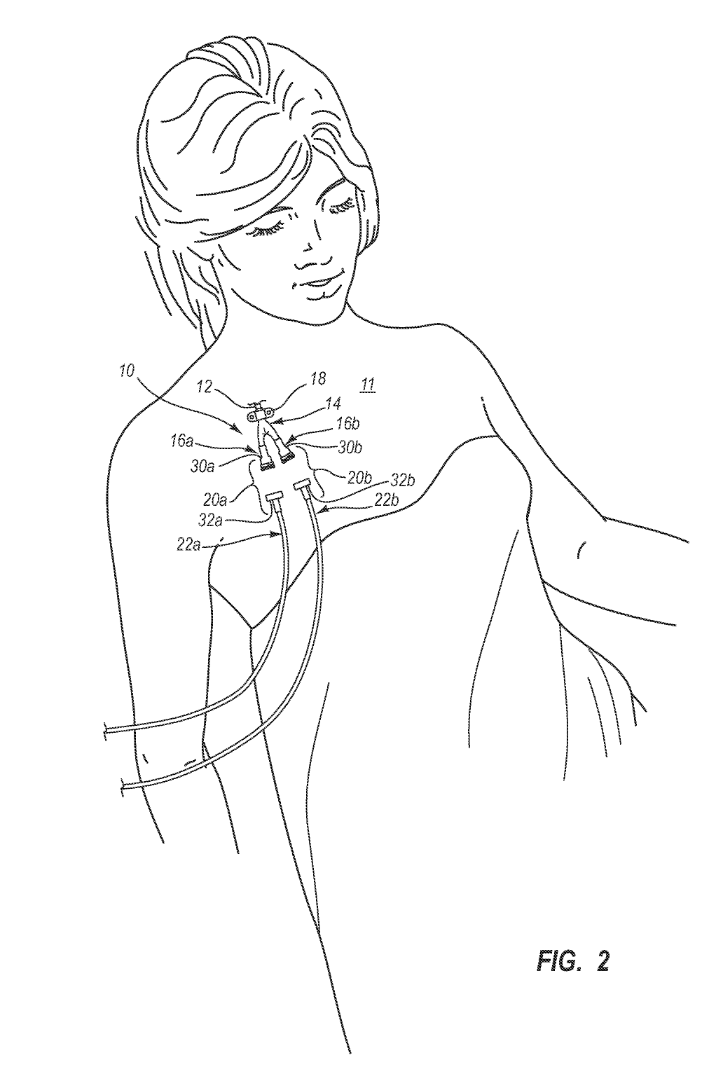

FIG. 2 is a perspective view of a dialysis catheter 10 which has been positioned within a chest of patient 11. In the illustrated embodiment, extension members 22a, b have been removed from connectors 20a, b of dialysis catheter 10. Connectors 20a, b comprise a male threaded member 30a, b and a female threaded member 32a, b. Female threaded members 32a, b are coupled to the distal ends of extension members 22a, b. When a practitioner desires to separate extension members 22a, b from the dialysis catheter 10, the practitioner simply removes the threaded coupling provided between male threaded members 30a, b and female threaded members 32a, b.

In the illustrated embodiment, dialysis catheter 10 may include a clamp configured to minimize the passage of blood from dialysis catheter 10 when female threaded members 32a, b have been separated from male threaded members 30a, b, such that relatively little blood issues from first arm 16a and second arm 16b of dialysis catheter 10. In this manner, a practitioner can attend to proper disposition of the extension members 22a, b without worrying about excessive blood loss from the components of dialysis catheter 10. By providing removal of extension members 22a, b at the end of a procedure, only transition member 14 and male threaded members 30a, b need remain attached to the patient on the exterior of the patient's body.

The relatively small and low profile of the combination of transition member 14 and male threaded members 30a, b, which remain connected on the chest of the patient between dialysis procedures, provides a comfortable and less intrusive dialysis catheter design. In this manner, accidental or inadvertent grasping of extension tubing or other components of the dialysis catheter are minimized or prevented. Additionally, the introduction of contaminants or other infectious agents that could be associated with extension members 22a, b which otherwise would remain attached to the patient is prevented. In the event that a practitioner has injected heparin or other medicaments into extension members 22a, b, the introduction of such medicaments into the patient in an undesired manner can be minimized by the simple removal of the extension members 22a, b from transition member 14.

As will be appreciated by those skilled in the art, a variety of types and configurations of connectors can be provided without departing from the scope and spirit of the present invention, as claimed. For example, according to one embodiment of the present disclosure, the components of the connector can remain secured to the transition member subsequent to removal of the extension members from the dialysis catheter. According to another embodiment of the present disclosure, mechanical coupling between the male threaded members and the female threaded members can be provided utilizing something other than a threaded coupling. According to yet another embodiment of the present disclosure, the extension members may be provided in a Y-connector such that a transition member of the dialysis catheter is not needed.

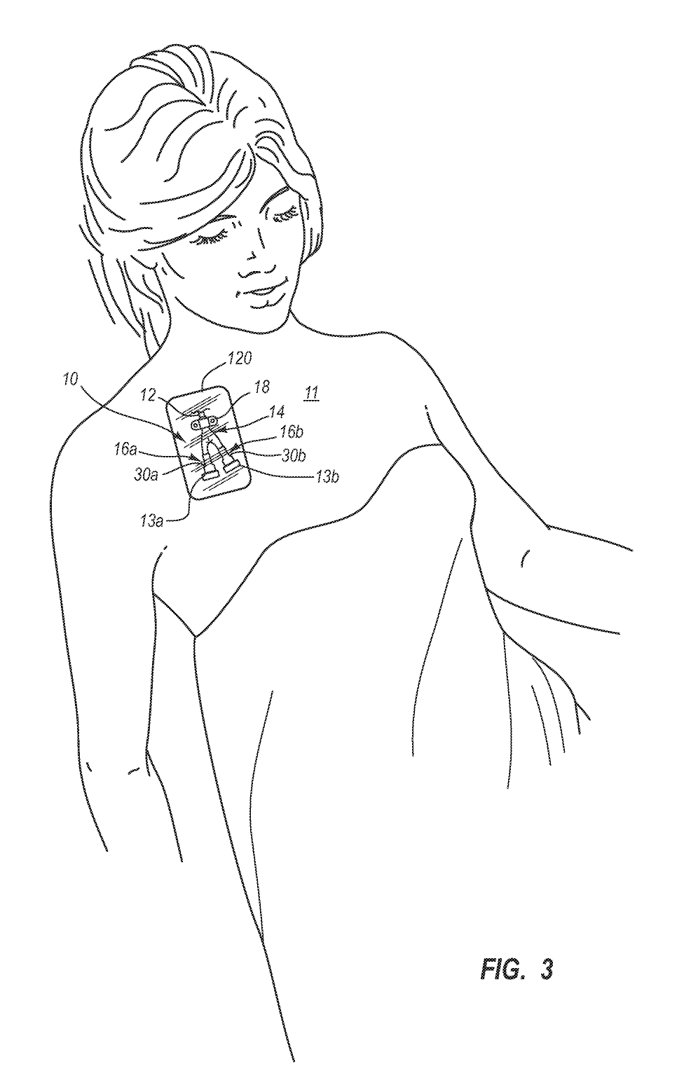

FIG. 3 is a perspective view of a dialysis catheter 10, according to one embodiment of the present disclosure. In the illustrated embodiment, dialysis catheter 10 includes a transition member 14, first arm and second arm 16a, b, and male threaded members 30a, b. Extension members 22a, b depicted in FIG. 2 have been fully removed from the dialysis catheter 10 such that only transition member 14 and first and second arms 16a, b remain positioned outside the patient. Additionally, removable caps 13a, b have been secured to male threaded members 30a, b to limit any blood or fluid leaks from the male threaded members 30a, b to the external environment. Additionally, removable caps 13a, b help to maintain the sterile nature of male threaded members 30a, b by providing a physical barrier between male threaded members 30a, b and the external environment. A practitioner may squeeze or clamp first and second arms 16a, b to stop the flow of blood during positioning of the removable caps 13a, b.

In the illustrated embodiment, a catheter protective layer 120 has been positioned over dialysis catheter 10. Catheter protective layer 120 comprises a covering, dressing or similar physical barrier, which is utilized to secure, cover or otherwise maintain catheter protective layer 120. Catheter protective layer 120 can comprise OPSITE™ which is a substantially transparent adhesive covering which can be secured to the skin of the patient. Catheter protective layer 120 secures dialysis catheter 10 in a desired position relative to the patient. In this manner, inadvertent moving or grasping of the dialysis catheter by the patient is minimized. Additionally, damage or injury to the catheter insertion site or accidental removal of the dialysis catheter from the patient is minimized. The substantially transparent nature of catheter protective layer 120 also permits a practitioner to view the catheter insertion site and the components of dialysis catheter 10. In this manner, the practitioner can ensure proper operation of the dialysis catheter and that any infection or damage that may occur at the catheter insertion site can be properly cared for or otherwise remedied.

Catheter protective layer 120 can minimize the introduction of contaminants or other infectious agents relative to the components of dialysis catheter 10. For example, once a practitioner has removed extension members 22a, b from engagement with male threaded members 30a, b, the practitioner can position removable caps 13a, b over male threaded members 30a, b. By maintaining sterile conditions during the positioning of cap members 13a, b over male threaded members 30a, b, subsequent placement of a catheter protective layer 120 over dialysis catheter 10 minimizes the potential for introduction of infection to the dialysis catheter. In this manner, the sterile conditions at both the dialysis catheter insertion site and with respect to male threaded members 30a, b after the completion of a dialysis procedure can be maintained.

As will be appreciated by those skilled in the art, a variety of types and configurations of catheter protective layers can be utilized without departing from the scope and spirit of the present invention, as claimed. For example, according to one embodiment of the present disclosure, the catheter protective layer can include a window which can be opened to facilitate cleaning of the catheter insertion site between dialysis procedures. According to another embodiment of the present disclosure, a material other than OPSITE™ can be utilized as a dressing or covering for the dialysis catheter. According to yet another embodiment of the present disclosure, the catheter protective layer can be utilized in connection with the male threaded members to minimize introduction of contaminants and can also be utilized in the place of removable caps.

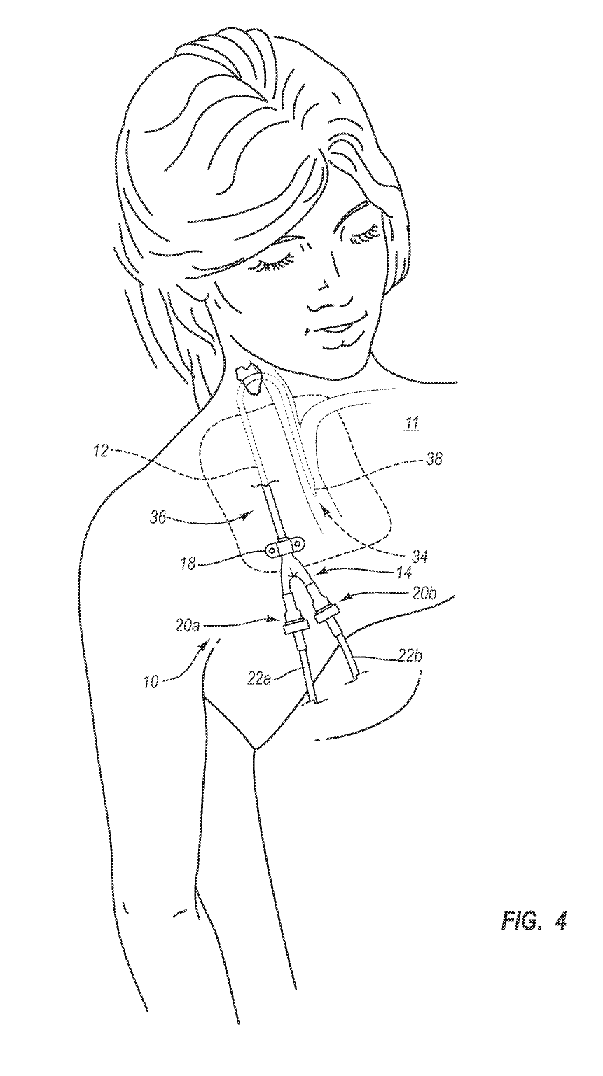

FIG. 4 is a perspective view of dialysis catheter 10 illustrating insertion of dialysis catheter 10 into a large vessel of patient 11 during a dialysis catheter insertion procedure. Access tube 12 of dialysis catheter 10 includes a distal end 34 and a proximal end 36. Distal end 34 includes a distal tip 38. In the illustrated embodiment, distal tip 38 has been threaded through the jugular vein, which is positioned within the patient's chest proximate to the patient's clavicle. Distal tip 38 has then been threaded from the jugular vein into a large vessel of the patient, in this case, the superior vena cava. In the depicted embodiment, distal end 34 is depicted during an insertion procedure such that the proximal components of dialysis catheter 10 are not in their desired position for securement of suture bracket 18 to the patient. In other words, distal end 34 has not been fully threaded to its desired position within the patient, transition member 14 is not positioned at a desired placement on the patient's chest and suture bracket 18 been not been sutured or otherwise secured to the patient's skin.

In the illustrated embodiment, extension members 22a, b are shown connected to connectors 20a, b for the sake of simplicity. As will be appreciated by those skilled in the art, extension members 22a, b can remain disconnected from connectors 20a, b during insertion of access tube 12 into the patient and/or during other aspects of the dialysis procedure. Additionally, a variety of insertion techniques can be utilized during placement of dialysis catheter 10 without departing from the scope and spirit of the present invention, as claimed. For example, in one embodiment, the access tube can be introduced through the patient's subclavian vein or femoral vein as opposed to being inserted through the jugular vein. However, the access tube 12 may be introduced via the external jugular or cephatic vein. According to yet another embodiment of the present disclosure, the distal end of access tube 12 can be positioned in another large vessel other than the superior vena cava.

In the illustrated embodiment, distal tip 38 of access tube 12 includes a predetermined profile. The profile of distal tip 38 facilitates the aspiration of blood from within the superior vena cava to the proximal portions of dialysis catheter 10. Similarly, distal tip 38 is configured such that once the blood has been treated in the hemodialysis machine, the blood can be returned to the patient through the return or delivery lumen of access tube 12 without interfering with the ongoing intake of blood through the intake lumen.

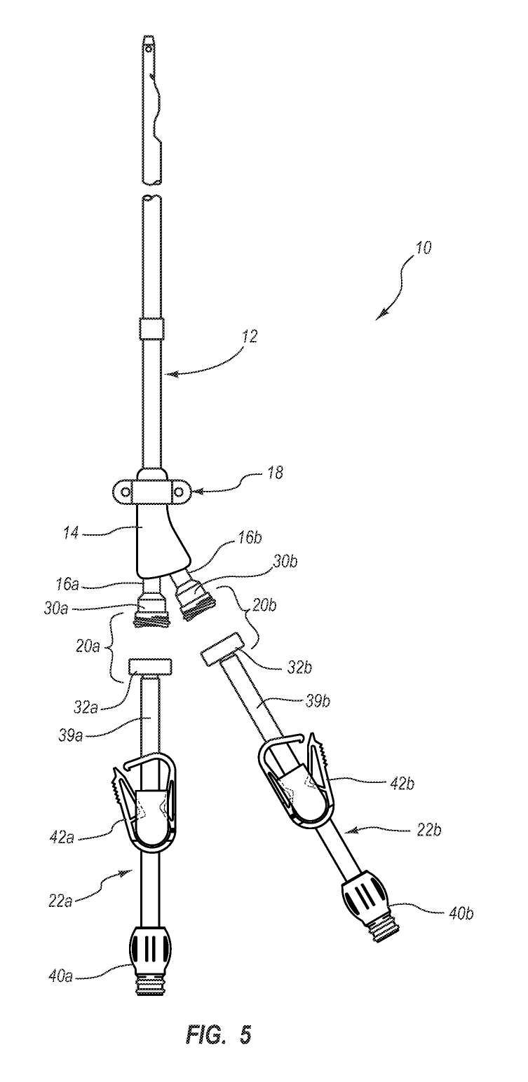

FIG. 5 is a perspective view of a dialysis catheter 10 according to one embodiment of the present disclosure. In the illustrated embodiment, extension members 22a, b are illustrated adjacent male threaded members 30a, b of dialysis catheter 10. Male threaded members 30a, b are coupled to first arm 16a and second arm 16b extending from transition member 14. In this manner, when access tube 12 is fully inserted into a patient and extension members 22a, b are removed from male threaded members 30a, b, only transition member 14, suture bracket 18, first arm 16a, second arm 16b, and male threaded members 30a, b are positioned externally on the patient.

In the illustrated embodiment, a first extension member 22a and a second extension member 22b are illustrated. Extension member 22a includes tubing 39a, luer coupler 40a, and clamp member 42a. Additionally, a female threaded member 32a may be positioned on the distal end of extension member 22a. Female threaded member 32a is configured to secure extension member 22a to male threaded member 30a of connector 20a. In this manner, extension member 22a can be secured to transition member 14 of dialysis catheter 10. When extension member 22a is secured to transition member 14 of dialysis catheter 10, extension member 22a may be positioned in fluid communication with first arm 16a and the associated lumen of access tube 12 of dialysis catheter 10. For example, in the event that first arm 16a corresponds with an intake lumen of access tube 12, blood can pass from intake lumen of access tube 12 into first arm 16a of transition member 14. The blood can then flow through male threaded member 30a, female threaded member 32a, and into extension member 22a.

In the illustrated embodiment, tubing 39a is secured to female threaded member 32a. Tubing 39a provides a conduit for the passage of fluids from female threaded member 32a into extension member 22a. Additionally, tubing 39a provides desired clearance between female threaded member 32a and the proximal portions of extension member 22a. According to one embodiment of the present disclosure, tubing 39a provides a flexible extension to provide clearance between the components to which dialysis catheter 10 is to be secured and transition member 14. Extension member 22a also includes a luer coupler 40a. Luer coupler 40a provides a threaded fitting to facilitate the coupling of extension member 22a to tubing associated with a hemodialysis machine. A clamp member 42a may also be provided. Clamp member 42a allows a practitioner to seal the lumen of tubing 39a until desired attachment of luer coupler 40a to the tubing associated with the hemodialysis machine is accomplished. Additionally, clamp member 42a can be actuated to seal the lumen of tubing 39a when a dialysis procedure is being completed or where a practitioner is preparing to separate luer coupler 40a from tubing associated with the hemodialysis machine. Once desired connection of luer coupler 40a has been effectuated, a practitioner can release clamp member 42a and allow the flow of blood through extension member 22a.

In the illustrated embodiment, second extension member 22b includes tubing 39b, luer coupler 40b, and clamp member 42b. Additionally, a female threaded member 32b may be positioned on the distal end of extension member 22b. Female threaded member 32b is configured to secure extension member 22b to male threaded member 30b of connector 20b. In this manner, extension member 22b can be secured to transition member 14 of dialysis catheter 10. When extension member 22b is secured to transition member 14 of dialysis catheter 10, extension member 22b may be positioned in fluid communication with second arm 16b and the associated lumen of access tube 12 of dialysis catheter 10. For example, in the event that second arm 16b corresponds with a delivery lumen of access tube 12, blood can pass from a hemodialysis machine into extension member 22b. From extension member 22b, the blood can then flow through female threaded member 32b to male threaded member 30b. From male threaded member 30b, the blood can flow from second arm 16b, through intake lumen of access tube 12, and into the patient.

In the illustrated embodiment, tubing 39b is secured to female threaded member 32b. Tubing 39b provides a conduit for the passage of fluids from female threaded member 32b into extension member 22b. Additionally, tubing 39b provides desired clearance between female threaded member 32b and the proximal portions of extension member 22b. According to one embodiment of the present disclosure, tubing 39b provides a flexible extension to provide clearance between the components to which dialysis catheter 10 is to be secured and the transition member 14. Extension member 22b also includes a luer coupler 40b. Luer coupler 40b provides a threaded fitting to facilitate the coupling of extension member 22b to tubing associated with a hemodialysis machine. A clamp member 42b may also be provided. Clamp member 42b allows a practitioner to seal the lumen of tubing 39b until desired attachment of luer coupler 40b to the tubing associated with the hemodialysis machine is accomplished. Additionally, clamp member 42b can be actuated to seal the lumen of tubing 39b when a dialysis procedure is being completed or where a practitioner is preparing to separate luer coupler 40b from tubing associated with the hemodialysis machine. Once desired connection of luer coupler 40b has been effectuated, a practitioner can release clamp member 42b and allow the flow of blood through extension member 22b.

As will be appreciated by those skilled in the art, a variety of types and configurations of extension members can be utilized without departing from the scope and spirit of the present invention, as claimed. For example, according to one embodiment of the present disclosure, extension members can be provided which do not utilize clamp members. According to another embodiment of the present disclosure, the tubing is connected directly from the hemodialysis machine to the transition member rather than utilizing an intermediary connection component. According to another embodiment of the present disclosure, couplings other than threaded couplings can be provided in connection with the luer couplings.

Various catheter types and tip designs may be used including split tip or step tip designs, infusion catheters, access catheters, non-tunneled catheters, and the like. Exemplary catheter lumen sizes for one embodiment of dialysis catheters may range from between 14 to 16 French. However, other lumen sizes may be used. Exemplary catheter lumen sizes for one embodiment of infusion catheters may range from between 7 to 10 French, while other lumen sizes may be used depending upon the particular catheter and application.

The present disclosure may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A catheter for use in accessing the vasculature of a patient, the catheter comprising:
an elongate access tube having a distal end, a proximal end, and a lumen extending therebetween, the distal end having a distal tip to be introduced into the patient's vasculature and the lumen allowing the passage of fluid between the distal end and the proximal end of the elongate access tube; and
a transition member having one or more connectors configured to be operably connected to a fluid delivery device, the one or more connectors allowing removable coupling and fluid communication between the lumen and the fluid delivery device.

2. The catheter of claim 1, wherein the elongate access tube comprises an intake lumen and a delivery lumen extending between the distal and proximal ends, such that the intake lumen allows passage of fluid from the distal end to the proximal end of the elongate access tube and the delivery lumen facilitates the delivery of fluid from the proximal end to the distal end of the elongate access tube.

3. The catheter of claim 2, wherein the catheter comprises a dialysis catheter such that the fluid delivery device is a hemodialysis machine, the transition member is configured to be operably connected to a hemodialysis machine, the intake lumen allows the passage of blood from the distal end to the proximal end of the elongate access tube and the delivery lumen facilitates the delivery of blood from the proximal end to the distal end allowing filtered blood to be returned to the patient's vasculature.

4. The catheter of claim 2, wherein the transition member includes a first arm and a second arm.

5. The catheter of claim 4, wherein the first arm is in fluid communication with the intake lumen of the elongate access tube and the second arm is in fluid communication with the delivery lumen of the elongate access tube.

6. The catheter of claim 5, wherein the one or more connectors comprise a first connector and a second connector.

7. The catheter of claim 6, wherein the first connector is coupled to the first arm of the transition member.

8. The catheter of claim 6, wherein the second connector is coupled to the second arm of the transition member.

9. The catheter of claim 6, wherein the one or more connectors comprise:
a male threaded member coupled to one of the transition member or one or more extension members that are configured to be connected to the fluid delivery device; and
a female threaded member coupled to the other of the transition member or the one or more extension members, wherein the male threaded member is configured to mate with the female threaded member to secure the one or more extension members to the transition member.

10. The catheter of claim 9, wherein the one or more extension members include a first end, which is secured to one of the male threaded member and the female threaded member, and a second end having a threaded coupling for securement to tubing which is coupled to the fluid delivery device.

11. A dialysis catheter comprising:
an elongate access tube configured to be positioned in a large vessel of a patient and having a first and second lumen for transmitting blood from patient to a hemodialysis machine and for returning blood from the hemodialysis machine to the patient;
a transition member for separating the first lumen from the second lumen into first and second arms of the transition member;
a connector for allowing connection of other components of the dialysis catheter or secondary apparatus to at least one of the first arm and second arm of the transition member allowing connection of at least one of the first lumen and second lumen of the elongate access tube to be placed in fluid communication with the hemodialysis machine.

12. The dialysis catheter of claim 11, further comprising an extension member configured to couple to the connector and configured to couple to tubing connected to the hemodialysis machine.

13. The dialysis catheter of claim 12, wherein the connector is configured to receive a cap to limit leakage through the connector when the connector is not couple to the extension member.

14. A dialysis catheter for use in accessing the vasculature of a patient and for transferring a volume of blood from a patient to a dialysis machine for filtering of the blood and returning the filtered blood to the patient, the dialysis catheter comprising:
an elongate access tube having a distal end, a proximal end, an intake lumen and a delivery lumen extending therebetween, the distal end having a distal tip to be introduced into the patient's vasculature and the intake lumen allowing the passage of blood from the distal end to the proximal end of the elongate access tube and the delivery lumen facilitating the delivery of blood from the proximal end to the distal end allowing filtered blood to be returned to the patient's vasculature;
a transition member secured to the proximal end of the elongate access tube, the transition member having a branch component having a first arm and a second arm, the first arm being in fluid communication with the intake lumen of the elongate access tube and the second arm being in fluid communication with the return lumen of the elongate access tube;
first and second connectors being secured to the first arm and second arm of the transition member, the first and second connectors having a threaded securement component for facilitating the securement of the first and second connectors to secondary apparatus; and
first and second extension members having threaded collars and configured to be secured to a dialysis machine or tubing associated with a dialysis machine.

15. The dialysis catheter of claim 14, wherein the first and second connectors are integrally coupled to the transition member.

16. The dialysis catheter of claim 14, wherein the first and second connectors comprise male threaded fittings coupled to the transition member.

17. The dialysis catheter of claim 16, wherein the first and second connectors include female threaded fittings coupled to the first and second extension members for securement to the male threaded fittings of the first and second connectors.
